(19) United States
(12) Patent Application Publication
(10) Pub. No.: US 2002/0138117 A1
(43) Pub. Date: Sep. 26, 2002

(54) Apparatus and method for selectively removing a body fat mass in human body

(76) Inventor: Young Tae Son, Seoul (KR)

Correspondence Address: Arent Fox Kintner, Plotkin & Kahn, Suite 400, 1050 Connecticut Avenue NW, Washington, DC, 20036-5339, US

(21) Appl. No.: 10/069,078

(22) PCT Filed: Mar. 22, 2001
(86) PCT No.: PCT/KR01/00455

(30) Foreign Application Priority Data
Jun. 21, 2000 (KR) 2000-34253

(51) Int. Cl. A61N 1/18

(52) U.S. Cl. 607/72

(57) ABSTRACT

The present invention relates to a method and apparatus for removing body fat. While an obesity patient or person wants to remove body fat built up in a certain part is conducting aerobic exercise such as running with attaching a surface-attaching type catheter on the fat part, the apparatus resolves and removes body fat by applying pulses, whose frequency is 10˜120 Hz, through the catheter, and changes the exercising condition based upon his or her heart rate and standard heart rate, thereby conducting efficient removal of body fat.

### 1. TECHNICAL FIELD

The present invention relates to method and apparatus for removing excessive fat mass in a human body, and more particularly, to apparatus and method for removing excess body fat in a target part of human body through the processes of calculating fatness ratio (FR) and setting and controlling an aerobic exercise criteria such as standard heart rate based on individual information such as age, weight, or height etc. while applying low-frequency pulses to a human body.

### 2. BACKGROUND ART

In general, fatness means that one's weight is above the standard. To be exact, it means that body fat has been over-accumulated in a human body. Standard weight for a person is obtained by multiplying 0.9 after subtracting 100 from his or her height.

Even though person's weight is above the standard, we will not say that he or she is fat if fat mass in his or her body is below the standard FR. Also, even though person's weight is below the standard, it may be possible to say that he or she is fat if fat mass in his or her body is above the standard FR.

Accordingly, FR to indicate how much fat mass has been accumulated in a human body is basis for determining obesity patient. Standard FR for a male is 25%, and 28˜30% for a female. If FR is above the standard, medical cure is needed to prevent various adult diseases.

The body fat mass can be measured accurately with a diagnostic apparatus such as ‘body composition analyzer’. Generally, body fat mass is mainly accumulated in an abdominal region of a human body, and such body fat mass can be easily flowed into blood, which causes various adult diseases such as hypertension, arteriosclerosis, diabetes, and hyperlipemia.

Accordingly, abdominal obesity of middle-aged persons is regarded as a ‘red signal’ warning cerebral apoplexy (palsy), myocardial infarction, etc. which might bring about sudden death. Basic method for curing obesity in oriental medicine is constitutional dietary treatment and medicinal therapy. Lately, natural therapies such as the ‘Ear Acupuncture Therapy’, in which ears are acupunctured to control appetite and incretion, and the ‘Aroma Therapy’, and so on are used besides the ‘Lipolysis Acupuncture’ for removing target parts of a human body.

However, because a target part, for example, abdominal region in which fat mass is easily over-accumulated has been acupunctured for a long time in the ‘Lipolysis Acupuncture’, an obesity patient may suffer from inconvenience. Also, the ‘Lipolysis Acupuncture’ has low efficacy in removing body fat because it cannot keep pace with aerobic exercises such as running which could double the efficacy.

### 3. DISCLOSURE OF INVENTION

It is an object of the present invention to provide method and apparatus for removing excessive fat mass in a human body, which applies low-frequency pulses to an obesity patient while he or she keeps practicing an aerobic exercise which can double fat removing efficacy.

It is another object of the present invention to provide method and apparatus for removing excessive fat mass in a human body, which controls operation of an athletic equipment such as a running machine based upon comparison of present heart rate of a user with a heart rate reference after calculating FR and setting an exercise criteria including heart rate reference automatically based on individual information such as age, weight, height, and so on.

An apparatus for removing excessive fat mass in a human body according to the present invention, comprises a generating means generating electric pulses of low-frequency band; and a transmitting means transmitting the electric pulses to an exercising person.

Another apparatus for removing excessive fat mass in a human body according to the present invention, installed in an athletic equipment for effectively removing body fat during exercise, comprises a generating means generating electric pulses of low-frequency band; a transmitting means transmitting the electric pulses to an exercising human body; and an attaching means for attaching the conducting means onto the human body.

Another apparatus for removing excessive fat mass in a human body according to the present invention, installed in an aerobic athletic equipment, comprises a measuring means for measuring present heart rate of an exercising person; a comparing means for comparing the measured heart rate with a preset heart rate reference; and a controlling means for controlling operation of the aerobic athletic equipment based on the comparison result of the comparing means.

Another apparatus for removing excessive fat mass in a human body according to the present invention, comprises a generating means generating electric pulses of low-frequency band; a transmitting means transmitting the electric pulses to an exercising person; an attaching means for attaching the transmitting means onto the exercising person, wherein the generating means, the transmitting means, and the attaching means are installed in an athletic equipment; a measuring means for measuring present heart rate of a person exercising on the athletic equipment; a comparing means for comparing the measured heart rate with a preset heart rate reference; and a controlling means for controlling operation of the athletic equipment based on the comparison result of the comparing means.

A method for removing excessive fat mass in a human body according to the present invention, generates electric pulses of low-frequency band, and transmits the electric pulses to an exercising person.

The present invention for removing excessive fat mass in a human body enables an obesity patient or a person, who wants to remove his or her fat mass, to keep practicing an aerobic exercise such as running for a long time with a plurality of pads attached onto his or her fat parts while electric pulses of frequency 10˜120 Hz, which resolves body fat through stimulus, are being applied through the attached pads.

The present invention for removing excessive fat mass in a human body also calculates user's FR and sets aerobic exercise criteria including heart rate reference automatically based on person's information on age, weight, height, and so on, and changes an exercising condition of an athletic equipment such as a running machine based upon comparison of his or her heart rate with the heart rate reference.

According to the present invention, body fat removal efficiency by an aerobic exercise can be doubled.

### 4. BRIEF DESCRIPTION OF DRAWINGS

The accompanying drawings, which are included to provide a further understanding of the invention, illustrate the preferred embodiments of the invention, and together with the description, serve to explain the principles of the present invention.

In the drawings:

FIG. 1 is a simplified block diagram of an apparatus for removing fat mass in a human body according to the present invention;

FIG. 2 is a pictorial representation showing a belt-type conducting means with pads for surface-attaching;

FIG. 3 is a flowchart of a method for removing fat mass in a human body according to the present invention;

FIG. 4 is an exemplary table showing entered individual information of a user and related aerobic exercise criteria set automatically based on the individual information;

FIG. 5 is a simplified block diagram of another apparatus for removing fat mass in a human body according to the present invention;

FIG. 6 is a flowchart of another method for removing fat mass in a human body in accordance with the present invention; and

FIG. 7 is another exemplary table showing entered individual information of a user and related aerobic exercise criteria and condition set automatically based on the individual information.

### 5. MODES FOR CARRYING OUT THE INVENTION

In order that the invention may be fully understood, preferred embodiments thereof will now be described with reference to the accompanying drawings.

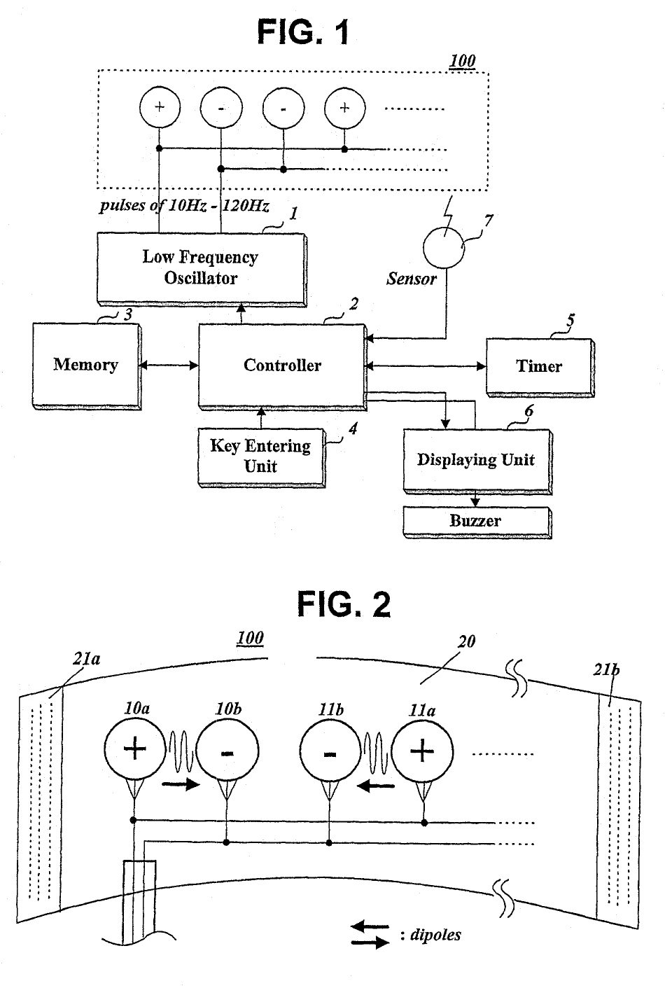

FIG. 1 is a simplified block diagram of an apparatus for removing body fat in a human body according to the present invention. The apparatus of FIG. 1 comprises a low-frequency oscillator 1 oscillating low-frequency pulses of 10˜120 Hz; a key entering unit 4 for entering individual information of a user such as age, weight, height, etc.; a controller 2 controlling operations of all elements, and calculating FR and setting aerobic exercise criteria such as heart rate reference for a person based on the entered individual information; a memory 3 for storing the calculated FR and the set heart rate reference, the entered individual information, and temporary data for controlling operations of all elements; a timer 5 for counting time set for control operation of the controller 2; a displaying unit 6 for presenting individual information entered from the key entering unit 4 and control status of the controller 2; a sensor 7 sensing heartbeats with its surface attached onto a heartbeat-detectable part of a human body; and a conducting unit 100 equipping with several fat-part attaching pads through which the low-frequency pulses from the low-frequency oscillator 1 are applied into target parts of a human body.

As shown on FIG. 2, the conducting unit 100 comprises a flexible flat belt 20 of which material is non-woven fabric for ventilation; a pair of Velcro tapes 21a and 21b attached at each side of the flat belt 20; and several surface-attaching pads 10a, 10b, . . . for conducting the low-frequency pulses into target fat parts.

The pads effectively conducts 10˜120 Hz pulses into a target fat part, for example, abdominal region in which fat mass is over-accumulated. The surface-attaching pads are fixed on the flat belt 20 with positive (+) and negative (−) pole paired. Each pair of pads is arranged at 5-cm intervals to equally apply the low-frequency pulses to a fat part and is also arranged such that each dipole moment of each pair should be alternated as shown in FIG. 2 to prevent frequency interference between neighboring pairs.

An obesity patient or a person who wants to remove his or her fat mass accumulated in a certain part spreads some gel onto a target part to decrease resistivity between pads and skin, and the conducting unit 100 is closely attached around the gel-spread part. Due to this tightly attachment, the person can practice an aerobic exercise such as running while body fat resolving treatment by low-frequency pulses is in progress.

When individual information of a user such as age, weight and height is entered through the key entering unit 4, the controller 2 calculates FR of the user and sets standard reference of heart rate (called ‘SRHR’ hereinafter) based on the entered individual information, and controls operations of all elements to effectively remove fat mass of the target part. The detailed operations for effectively removing body fat are as follows.

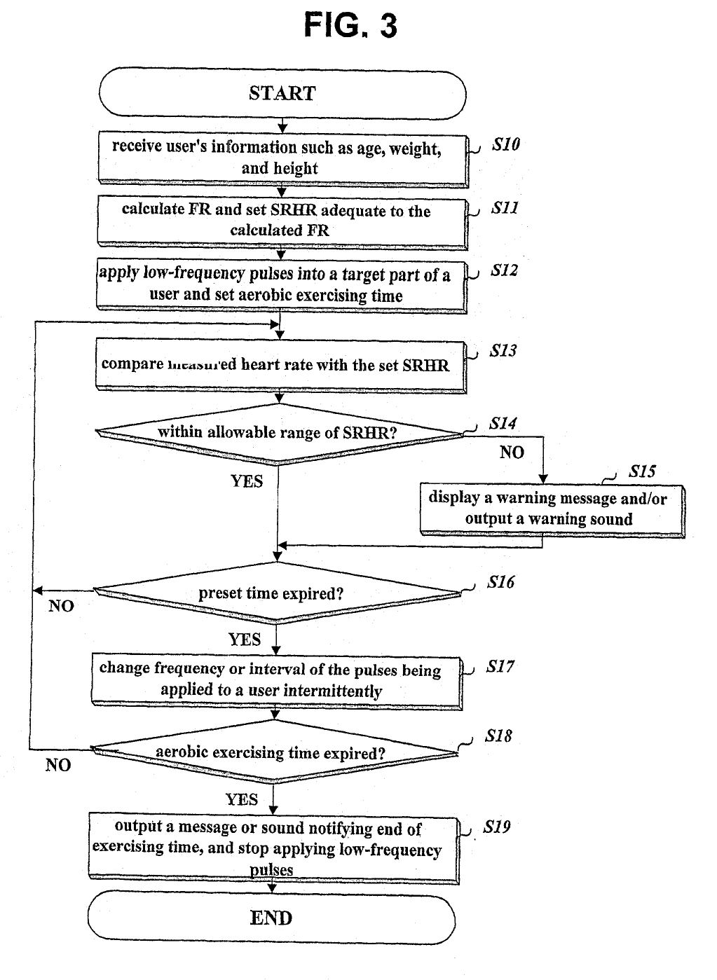

FIG. 3 is a flowchart of a method for removing fat mass in a human body in accordance with the present invention. When a user enters his or her individual information such as age, weight, and height (S10) through the key entering unit 4, the controller 2 calculates FR of the user and sets SRHR adequate to the calculated FR using the following equations (S11).

SRHR=Standard Heart Rate by Age−α=((220−entered Age)×0.6)−60 , where α is a heart rate decrement factor (HRDF) according to FR Eq. (1)

FR=((entered Weight−Standard Weight)/(Standard Weight))×100 Eq. (2)

(Standard Weight by Height=(entered Height−100)×0.9 Eq. (3)

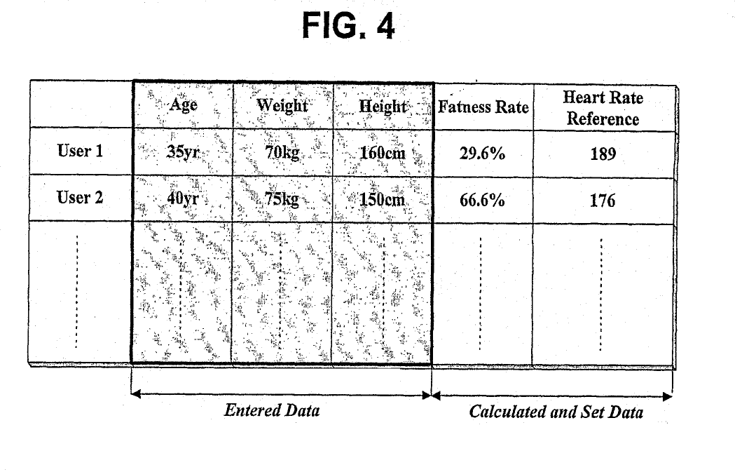

For example, as shown in FIG. 4, if an entered weight of a user is 70 kg and height is 160 cm, the standard weight classified by height is calculated to 54 kg based on the given Eq. (3), and FR is calculated to 29.6% based on the given Eq. (2) because ((70−54)/54)×100=29.6. In general, not more than 10% of the standard weight is regarded as normal weight, and 10% to 20% is regarded as overweight. More than 20% is regarded as obesity.

And, if entered user's age is 35, the standard heart rate of that age is calculated to 111 based on the Eq. (1), namely, (220−35)×0.6. At that time, the HRDF α=20 predefined for that FR is subtracted from the calculated standard heart rate, therefore, the SRHR for the user is determined to 91.

The HRDF α is pre-specified in consideration of the fact that heart rate of an obesity patient becomes higher than that of normal person even though they are doing same aerobic exercise such as running. The pre-specified HRDF α may not be subtracted if a user choose to do that. The FR and SRHR calculated as above are stored in the memory 3 and also displayed on the displaying unit 6 as characters, therefore a user can check his or her FR and SRHR easily.

After that, the controller 2 drives the low-frequency oscillator 1 to apply low-frequency pulses through several pads 10a,10b,11a, 11b, . . . of the belt-type conducting unit 100 which are stick to a target part, for example, abdominal region in which fat mass is over-accumulated, and controls the timer 5 to set aerobic exercising time, for example, 20 minutes (S12).

The aerobic exercising time is set in consideration of the time of carbohydrate decomposition preceding decomposition of accumulated body fat mass. The aerobic exercising time may be set differently according to the FR and SRHR calculated based on the individual information entered through the key entering unit 4.

The controller 2 counts heartbeat signals outputted from the clip-shaped sensor 7 which is stick to a certain part such as an ear of a user, and measures the current heart rate of a user. After that, the controller 2 compares the measured heart rate with the set SRHR (S13), and if the difference exceeds the SRHR more than a predetermined allowable range (S14), for example, 10% of SRHR, it displays a warning message on the displaying unit 6 and/or outputs a warning sound through a buzzer (S15).

Accordingly, a user can perform aerobic exercise such as running more fully within the warning range. When a user feels less stimulus of the low-frequency pulses after a while, for example, 30 seconds (S16), the controller 2 controls the low-frequency oscillator 1 to change frequency of the pulses being applied to the conducting unit 100, or pulse interval intermittently (S17).

When the set aerobic exercising time expires (S18) while the controller 2 conducts the above-explained operations, the controller 2 outputs a message or sound notifying end of exercising time, and stops the oscillating operation of the low-frequency oscillator 1 (S19).

Accordingly, a user can practice an aerobic exercise such as running continuously while he or she undergoes medical treatment of removing body fat by pulses of low-frequency band applied through his or her wearing belt-type conducting unit with several pads tightly wrapping the fat part, therefore, efficiency of body fat removal is doubled.

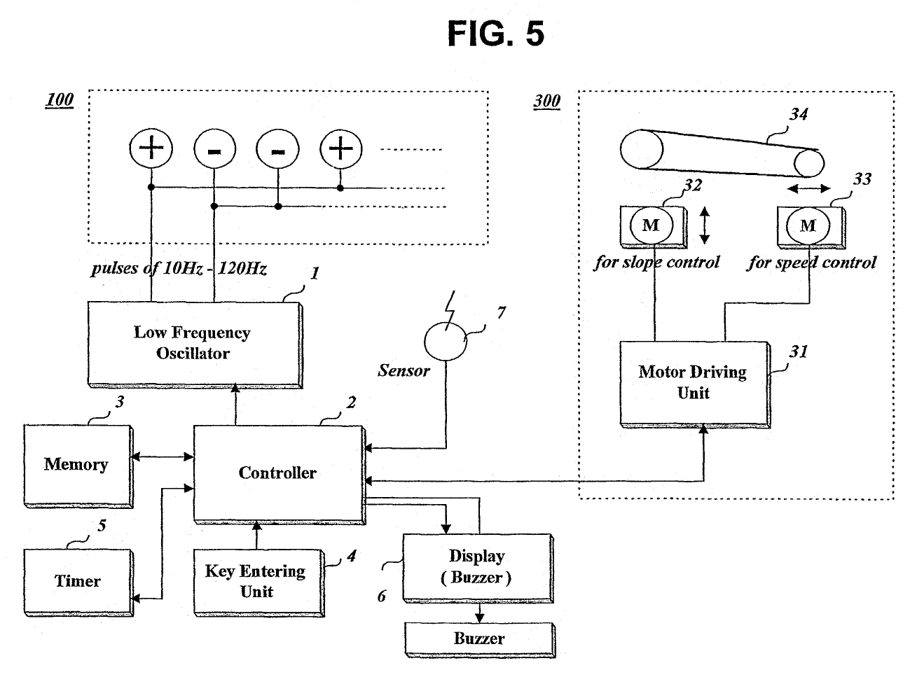

FIG. 5 is a simplified block diagram of another apparatus for removing body fat in a human body according to the present invention. The apparatus of FIG. 5, especially integrated into a running machine for an indoor aerobic exercise such as running, comprises a low-frequency oscillator 1 oscillating low-frequency pulses of 10˜120 Hz; a key entering unit 4 for entering individual information of a user such as age, weight, height, etc.; a controller 2 controlling operations of all elements, and calculating FR and setting aerobic exercise criteria such as SRHR for a user based on the entered individual information; a memory 3 for storing the calculated FR and the set SRHR, the entered individual information, and temporary data for controlling operations of all elements; a timer 5 for counting time set for control operation of the controller 2; a displaying unit 6 for presenting individual information entered from the key entering unit 4 and control status of the controller 2; a sensor 7 sensing heartbeats with its surface attached onto a certain heartbeat-detectable part of a human body; and a conducting unit 100, to be wrapped around a fat part of a human body, equipping with several fat-part attaching pads through which the low-frequency pulses from the low-frequency oscillator 1 are conducted into target parts of a human body.

The controller 2 controls a motor driving unit 31, installed in a running machine 300, which drives both a motor 33 for circulating a belt board 34 and another motor 32 for adjusting slope of the belt board 34.

Same as the previous description referring to the apparatus of FIG. 2, the conducting unit 100, wrapped tightly around a target part of user's body, conducts pulses of 10˜120 Hz oscillated from the low-frequency oscillator 1 into target fat parts of a user.

An obesity patient or a person who wants to remove his or her fat mass accumulated in a certain part spreads some gel onto a target part to decrease resistivity between pads and skin, and the conducting unit 100 is closely wrapped around the gel-spread part. Due to this tightly attachment, the person can practice an aerobic exercise such as running while body fat resolving treatment by low-frequency pulses is in progress.

When individual information of a user such as age, weight and height is entered through the key entering unit 4, the controller 2 calculates FR of the user and sets SRHR, aerobic exercise condition such as running speed, etc. based on the entered individual information, and controls operations of all elements to effectively remove fat mass of a target part.

The above-described body fat removing apparatus may be integrated into a running machine, or it may be manufactured as a separate control box to be connected to a side of a running machine.

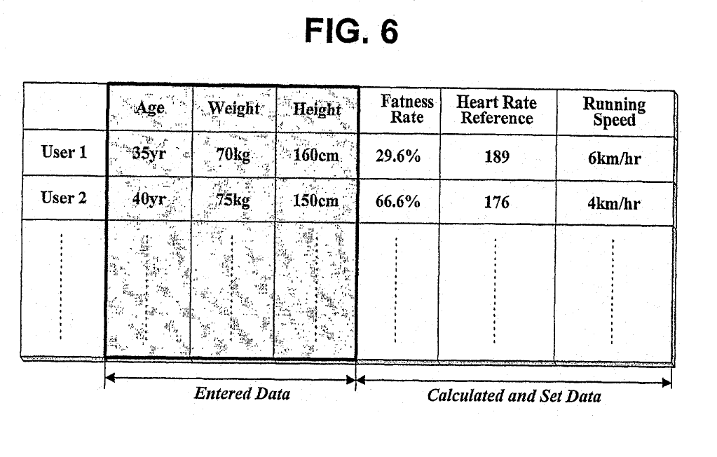

FIG. 6 is a flowchart of another method for removing fat mass in a human body in accordance with the present invention.

Same as explained above for the former embodiment, when a user enters his or her individual information such as age, weight, and height (S30) through the key entering unit 4 of the body fat removing apparatus connected with the running machine 300, the controller 2 calculates FR of the user and sets SRHR and running speed adequate to the calculated FR using the aforementioned Eqs. (1) to (3) (S31).

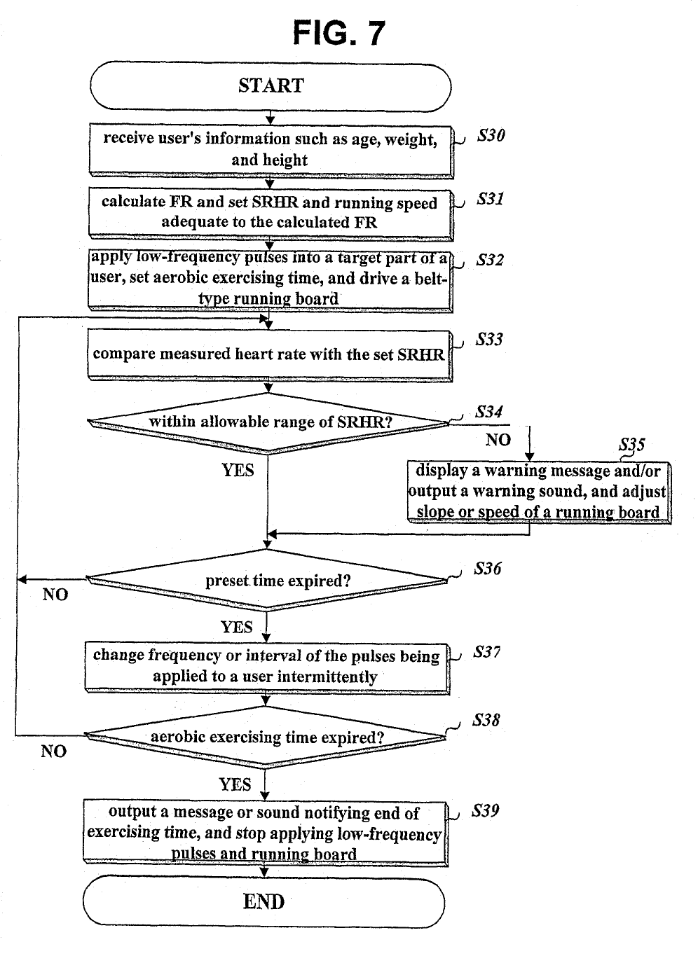

For example, as given in FIG. 7, FR of a user is derived based on his or her weight and height, and SRHR for that age is calculated based on entered his or her age. After that, HRDF α for the derived FR is subtracted from the calculated SRHR to decrease the SRHR of the user, if he or she is determined to obesity, to appropriate value. In addition, the running speed of the running machine 300 is set in inverse proportion to the derived HR of the user.

Running speed of the running machine 300 may have pre-defined discrete speed levels, so that one of them is chosen and set based on FR of an obesity patient. The FR, SRHR, and aerobic condition of running speed to be set as above are stored in the memory 3 and are also displayed on the displaying unit 6 as characters, therefore a user can recognize his or her FR, SRHR; and running speed, easily.

After that, the controller 2 drives the low-frequency oscillator 1 to apply low-frequency pulses through the several pads 10a,10b,11a,11b, . . . of the belt-type conducting unit 100 wrapped tightly around a target part, for example, abdominal region in which fat mass is over-accumulated, and it sends time data of aerobic exercise such as running to the timer 5 to set aerobic exercising time, for example, 20 minutes (S32).

The controller 2 counts heartbeat signals outputted from the clip-shaped sensor 7 which is stick to a certain part of a user such as an ear, and measures the current heart rate of a running user. While measuring current heart rate, the controller 2 compares the measured heart rate with the set SRHR (S33), and if the difference exceeds the predefined allowable range (S34), it displays a warning message on the displaying unit 6 and/or outputs a warning sound through a buzzer and controls the motor driving unit 31 of the running machine 300 to adjust moving speed or slope of the circulating belt board 34 at the same time (S35).

For example, the controller 2 compares the measured heart rate with the set SRHR, and, if the measured heart rate is below the set SRHR more than the predefined allowable range, it displays a warning message on the displaying unit 6 and/or outputs a warning sound through a buzzer, and, at the same time, increases slope or moving speed of the motor-driven belt board 34 through controlling the motor driving unit 31 of the running machine to increase strength of exercise.

If the difference is above the set SRHR more than the predefined allowable range, the controller 2 also displays another warning message on the displaying unit 6 and/or outputs another warning sound through the buzzer, and decreases slope or moving speed of the belt board 34 to decrease strength of exercise.

When a user feels less stimulus of the low-frequency pulses after a while, for example, 30 seconds (S36), the controller 2 controls the low-frequency oscillator 1 to change frequency of the pulses being applied to the conducting unit 100, or pulse interval intermittently (S37).

When the set aerobic exercising time expires (S38) while the controller 2 conducts the above-explained operations, the controller 2 outputs a message or sound notifying end of exercising time, and stops the operation of the low-frequency oscillator 1 and the motor-driven belt board 34 of the running machine 300 gradually (S39).

Accordingly, a user pan keep practicing an aerobic exercise such as running while he or she undergoes medical treatment of removing body fat by pulses of low-frequency band being applied through his or her wearing belt-type conducting unit with several pads tightly wrapping the fat part, therefore, body fat can be removed much more efficiently due to exercise and concurrent pulse applying.

The planar shape of the surface-attaching pads 10a, 10b, . . . may be round or rectangular to be attached tightly onto target parts of a human body. The surface-attaching pads may be equipped in a flexible long band, an abdominal binder, or a suit-type conducting unit, of which material is non-woven fabric for ventilation, besides the belt-type conducting unit 100, in order to apply low-frequency pulses oscillated from the low-frequency oscillator 1 into fat mass accumulated in a human body.

Each aerobic exercise criteria and condition set based on individual information may be stored as an item of a table in the memory 3. After this tabulation, aerobic exercise criteria and condition corresponding to input individual information can be chosen among items of the table and be set without calculation. Or if a user presses a special key on the key entering unit 4, an aerobic exercise criteria and condition defined in connection with the special key can be set through mechanical or electronic operation, which may simplify the structure of the body fat removing apparatus. Also, the apparatus for removing body fat in a human body may be implemented without the low-frequency oscillator 1 and the conducting unit 100 in case that it is linked with an exercising machine such as a running machine.

Furthermore, the apparatus for removing body fat in a human body may be equipped in another exercising machine, namely, a stepper or a bicycle.

The invention may be embodied in other specific forms without departing from the sprit or essential characteristics thereof. The present embodiments are therefore to be considered in all respects as illustrative and not restrictive, the scope of the invention being indicated by the appended claims rather than by the foregoing description and all changes which come within the meaning and range of equivalency of the claims are therefore intended to be embraced therein.

1. An apparatus for removing body fat in a human body, comprising:
a generating means generating electric pulses of low-frequency band; and
transmitting means transmitting the electric pulses to an exercising human body.

2. The apparatus set forth in claim 1, wherein said transmitting means comprises surface-attaching pads.

3. The apparatus set forth in claim 2, wherein the surface-attaching pads consist of a plurality of pairs of positive (+) and negative (−) pole pads, each pair being arranged at predetermined intervals, and also arranged such that each dipole moment of each pair should be alternated.

4. The apparatus set forth in claim 1, wherein said generating means changes the frequency band of the electric pulses at intervals, or changes pulse interval intermittently.

5. A body fat removing apparatus installed in an athletic equipment, comprising:
a generating means generating electric pulses of low-frequency band;
a transmitting means transmitting the electric pulses to an exercising human body; and
an attaching means for attaching the transmitting means onto the exercising human body.

6. The apparatus set forth in claim 5, wherein said attaching means is a belt, a long band, an abdominal binder, or a suit which is made of flexible material.

7. The apparatus set forth in one of claims 1 to 5, further comprising:
a measuring means for measuring heart rate of the human body; and
a comparing means for comparing the measured heart rate with a preset heart rate reference.

8. The apparatus set forth in claim 5, further comprising:
a measuring means for measuring heart rate of the human body;
a comparing means for comparing the measured heart rate with a preset heart rate reference; and
a controller for controlling operation of said athletic equipment based on the comparison result of said comparing means.

9. A method for removing body fat in a human body, comprising the steps of:
generating electric pulses of low-frequency band; and
transmitting the electric pulses to an exercising human body.

10. The method set forth in claim 9, further comprising the steps of:
measuring heart rate of the human body; and
comparing the measured heart rate with a preset heart rate reference.

11. The method set forth in claim 10, further comprising the step of controlling operation of an athletic equipment based on the result of said comparing step.

12. The method set forth in claim 11, wherein said controlling step controls the exercising speed and/or slope and/or exercising time of the athletic equipment.

13. The method set forth in claim 10 or 11, further comprising the step of outputting a warning message or a warning sound based on the result of said comparing step.

14. The method set forth in claim 10, wherein the heart rate reference is determined based on an age and/or maximum heart rate and/or fatness ratio of the human body.

15. A body fat removing apparatus installed in an aerobic athletic equipment, comprising:
a measuring means for measuring heart rate of a user;
a comparing means for comparing the measured heart rate with a preset heart rate reference; and
a controller for controlling operation of said athletic equipment based on the comparison result of said comparing means.

16. An apparatus for removing body fat in a human body, comprising:
a generating means generating electric pulses of low-frequency band;
a transmitting means transmitting the electric pulses to an exercising human body;
an attaching means for attaching the conducting means onto the exercising human body, wherein said generating means, said transmitting means, and said attaching means are installed in an athletic equipment;
a measuring means for measuring current heart rate of a person exercising on the athletic equipment;
a comparing means for comparing the measured heart rate with a preset heart rate reference; and
a controller for controlling operation of the athletic equipment based on the comparison result of the comparing means.

17. The apparatus set forth in claim 15 or 16, wherein the heart rate reference is determined based on an age and/or maximum heart rate and/or fatness ratio of the person.

18. The apparatus set forth in claim 15 or 16, wherein said controller controls the exercising speed and/or slope and/or exercising time of the athletic equipment.

19. The apparatus set forth in claim 15 or 16, further comprising an alarming means for outputting a warning message or a warning sound based on the comparison result of said comparing means.
